(19) United States
(12) Patent Application Publication
(10) Pub. No.: US 2003/0208780 A1
(43) Pub. Date: Nov. 6, 2003

(54) Recombinant constructs encoding T cell receptors specific for human HLA-restricted tumor antigens

(76) Inventors: Linda A. Sherman, La Jolla, CA (US); Joseph Lustgarten, La Jolla, CA (US)

Correspondence Address: EDWARDS & ANGELL, LLP, P.O. BOX 9169, BOSTON, MA, 02209, US

(21) Appl. No.: 09/774,681

(22) Filed: Feb. 1, 2001

Related U.S. Application Data
(62) Division of application No. 08/812,393, filed on Mar. 5, 1997.
(60) Provisional application No. 60/012,845, filed on Mar. 5, 1996.

(51) Int. Cl. A01K 67/00; C12P 19/34

(52) U.S. Cl. 800/6; 435/91.2

(57) ABSTRACT

Methods are described to obtain nucleic acid molecules that encode T cell receptors and their derivatives that are human HLA-restricted and which are specific for tumor-associated antigens found in human tumors. These nucleic acids are useful in preparing recombinant cells for diagnosis and therapy of human tumors.

### TECHNICAL FIELD

The invention is directed to recombinant T cell receptors and modified forms thereof that are useful in identifying displayed tumor antigens and in antitumor therapy.

### BACKGROUND ART

Cytotoxic T lymphocytes (CTLs) form an essential part of an immune response to infectious agents and to malignancies. Thus, CTLs which are directed to established tumors may be effective in destroying these targets. Greenberg, P. D. Adv Immunol (1991) 49:281-355. CTL may also be used to identify tumor-specific antigens such as MAGE, GP100, tyrosinase, and MART, as well as broadly expressed tumor-associated antigens such as P53 (Yanuck, M. et al. Cancer Res (1993) 53:3257-3261); Houviers, J. G. A. et al. Eur J Immunol (1993) 23:2072-2077; Her-2/neu (Peoples, G. E. et al. Proc Natl Acad Sci USA (1995) 92:432-436; Fisk, B. et al. J Exp Med (1995) 181:2109-2177; as well as the tumor antigen Ras (Skipper, J. et al. J Exp Med (1993) 177:1493-1498).

It has been typical that such tumor-specific CTLs have been obtained from tumor infiltrating lymphocytes (TILs). However, this is subject to a number of disadvantages due to the complexity of the system and the endogenous mechanisms to counteract the effect of these CTLs. Importantly, the most effective CTLs may have been eliminated (Schwartz, R. H. Cell (1989) 57:1073-1081); the target tumors may have become resistant (Browning, M. J. et al. Curr Opin Immunol (1992) 4:613-618); or the T cells may lose functional activity by down-regulating expression of the ζ chain of the CD3 complex or the p⁵⁶ LCK molecules (Mizoguchi, H. et al. Science (1992) 258:1795-1798).

In order to overcome these disadvantages, the present applicants have used transgenic mice as a source of CTLs that contain the desired nucleotide sequences encoding TCRs specific for tumor-associated antigens restricted by human HLAs. Both humans and HLA-A2 transgenic mice select the same A2-restricted antigenic epitopes from influenza (Vitiello, A. et al. J Exp Med (1991) 173:1007-1015). Also, the present applicants have shown that HLA-A2 transgenic mice can produce p53-specific, A2 restricted CTLs when immunized with certain p53 derived peptides. Theobald, M. et al.. Proc Natl Acad Sci USA (1995) 92:11993-11997.

Of course, if murine-derived TCRs are to be used in a human context, humanization of such TCRs would be advantageous. In order to avoid competition for dimerization with endogenous Vα/Cα or Vβ/Cβ TCR, it may be advantageous to prepare chimeric TCRs using the ζ region of the CD3 receptor as the transmembrane and cytoplasmic domain. Such constructs could be prepared in either dimeric or single-chain form. Competition by Vα/Cα or Vβ/Cβ for each other or for the availability of CD3 chains has already been shown by Gorochov, International J Cancer (1992) 8:53-57 and by Wegener, A. M. K. et al. Cell (1992) 68:83. Chimeric Vα/ζ÷Vβ/ζ chimeras were described by Engel, I. et al. Science (1992) 256:1318 who also showed that such chimeras could be activated by exposure to the appropriate antigen-MHC complex. In addition, Irving, B. A. et al. Cell (1991) 64:891 reported that chimeric molecules composed of the CD8/ζ or CD16/ζ and expressed in T cells had the capacity to transduce activation signals for IL-2 production and mediated specific cell lysis in a manner indistinguishable from those generated by the TCR/CD3 complex. In addition, Chung, S. et al. Proc Natl Acad Sci USA (1994) 91:12654-12658 constructed a single-chain TCR (scTCR) using the ζ-chain of CD3 and expressed it in T cells, thus conferring the T cells with the relevant specificity. These T cells further produce IL-2 on activation with the specific antigen. The present applicants have further confirmed this approach using clone 4 TCR as a model system.

However, there remains a need for a convenient source of nucleic acids encoding TCR molecules and their modified forms which are human HLA restricted and specific for common tumor-associated antigens. The present invention supplies this need.

### DISCLOSURE OF THE INVENTION

The invention provides materials that are useful in tumor diagnosis and therapy by permitting altered T lymphocytes to recognize and destroy unwanted tumor tissue. T cell receptor-encoding nucleic acid molecules can be obtained by immunizing transgenic mice which produce human HLA with tumor-associated antigens and recovering the nucleic acids encoding the T cell receptors from the cytotoxic T lymphocytes (CTL).

Thus, in one aspect, the invention relates to a method to prepare an isolated nucleic acid molecule comprising a nucleotide sequence encoding at least one of the variable regions of the α and β chains of a non-human TCR which TCR is human HLA-restricted and specific for a tumor-associated antigen, which method comprises cloning or amplifying a nucleic acid molecule containing said encoding nucleotide sequence from the CTL prepared by a method which comprises immunizing a transgenic non-human vertebrate which is modified so as to express at least one human HLA antigen with said tumor-associated antigen (TAA) so as to effect the production in said mouse of cytotoxic T lymphocytes which display human HLA-restricted TCR specific for said TAA and which contain nucleic acid molecules comprising nucleotide sequences encoding the α and β chain of said TCR and recovering the CTL.

In other aspects, the invention relates to nucleic acid molecules obtained by the foregoing method and to constructs employing their variable regions, to cells displaying TCRs or derivatives encoded by said nucleic acids or their modified forms, and use of these materials in diagnosis and therapy of human tumors.

### BRIEF DESCRIPTION OF THE DRAWINGS

FIG. 1 shows the structure of several derivatives of effective T cell receptors wherein the ζ region is substituted as a chimeric transmembrane and cytoplasmic region.

FIG. 2 shows, in more detail, the construction of the nucleotide sequence encoding such derivatives.

FIG. 3 shows the complete nucleotide sequence and deduced amino acid sequence of a single chain TCR derivative which contains variable α and β specific for HA linked through a short peptide linker and then fused through a CD8 hinge to the ζ chain.

FIG. 4 shows the ability of cells transfected with various modified TCR forms specific for HA to produce IL2 in response to stimulation with HA.

FIG. 5 shows the ability of CTL's generated in mice in response to Her 2/neu-peptides H3 and H7 to mice H7 or H3 bearing targets. CTLs from both A2.1×K^{b}×CD8 and from A2.1 transgenic mice were comparable in result.

FIG. 6 shows the sequence of various primers useful in cloning or amplifying the nucleotide sequences in coding during variable regions of α and β TCR chains.

FIGS. 7A and 7B show the nucleotide sequence and deduced amino acid sequence of the variable regions of the α and β chains of H7-specific TCR respectively.

FIG. 8 shows a diagram of an expression vector suitable for producing the modified TCRs of the invention.

FIG. 9 shows the ability of H7 specific modified TCR forms transfected in the 27J cells to effect IL2 production in said cells in response to the H7 peptide when the H7 peptide is presented in the presence of JA2 cells.

FIG. 10 shows the ability of the various modified H7 specific TCR constructs to stimulate IL2 production in 27J cells in response to tissues bearing Her2/neu-peptides.

### MODES OF CARRYING OUT THE INVENTION

The invention provides a convenient source for desirable recombinant materials that are useful in therapeutic and diagnostic procedures related to human tumors. Specifically, the materials of the invention provide a means whereby enhanced populations of cells that display appropriate TCRs for identifying and destroying tumor tissue may be obtained, as well as providing cells that are useful in evaluating the tumor-associated antigen that could usefully be targeted.

Briefly, the recombinant materials are obtained from CTL produced by immunizing nonhuman subjects with tumor-associated antigens associated with human tumors, where the nonhuman subject has been modified so as to be capable of expressing a human HLA. Thus, the relevant TCRs are not only specific for the human tumor-associated antigen, but also restricted by a human HLA. While murine subjects are clearly the most convenient at the present time, further developments in the construction of transgenic animals may permit alternative nonhuman subjects to be used equally conveniently in the near future. Such additional nonhuman subjects may include rats, avian subjects, larger mammals, or any appropriate vertebrate system that can be manipulated to provide it with human HLA and which can mount an immune response to provide CTLs with the appropriate T cell receptors.

Further, while the human HLA illustrated herein is A2, there is no theoretical reason why other HLA domains such as A1, A3, and B7 could not be used as well. Because transgenic mice are readily available which produce this antigen, the use of a A2 as the restrictive antigen is simply a matter of convenience. In addition, if murine subjects are used, and the MHC region is entirely human, it is preferred to use mice transgenic so as to express human CD8 as well as human Class MHC antigen. This is due to the inability of murine CD8 to interact effectively with human A2.1. Thus, expression of human CD8 on the murine cells facilitates lysis of target antigen presenting cells. On the other hand, for mice transgenic for MHC human/mouse chimeras, such as A2K^{b} mice also exemplified below, the presence of human CD8 is not necessary.

The recombinant materials relevant to the invention include those associated with the TCR produced by the nonhuman subject per se, and also derivatives of this TCR which retain their HLA restriction and specificity characteristics. Such derivatives contain the variable regions of the α and β chains either as dimers or in single chain form and are more advantageous than the nonhuman TCR per se for a number of reasons. First, if the desired TCR can be “humanized,” less unwanted side-reactions can be expected. Second, economies of production can be effected if shorter peptides can be substituted for the TCR per se. Third, if the TCR is produced as a single chain, rather than in its customary dimeric form, economies of production and ease of association of the relevant variable units are achieved. In all cases, substituting a derivative for one or both of the α and β chains or a single-chain form containing variable regions of both α and β precludes the formation of hybrid TCRs wherein for example the desired TCR α chain is coupled with an endogenous TCR β. Thus, the recovery of cells which produce the desired derivative is greater.

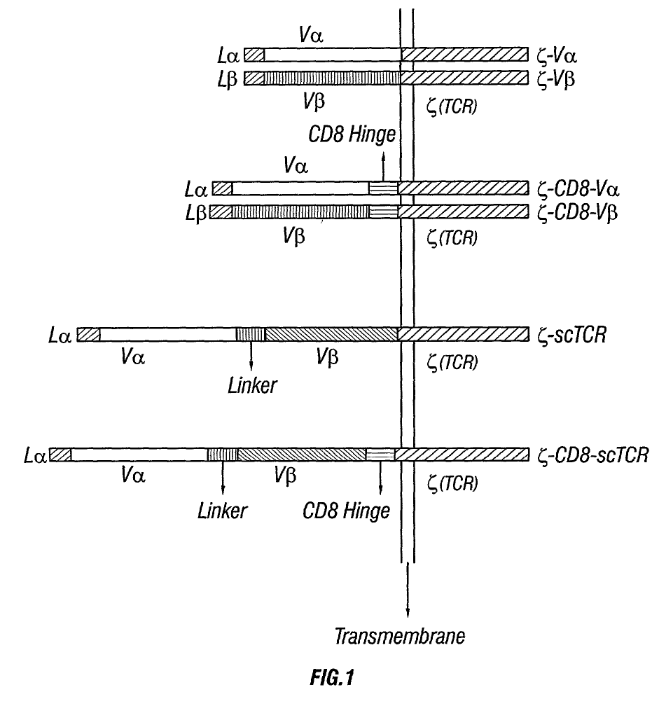

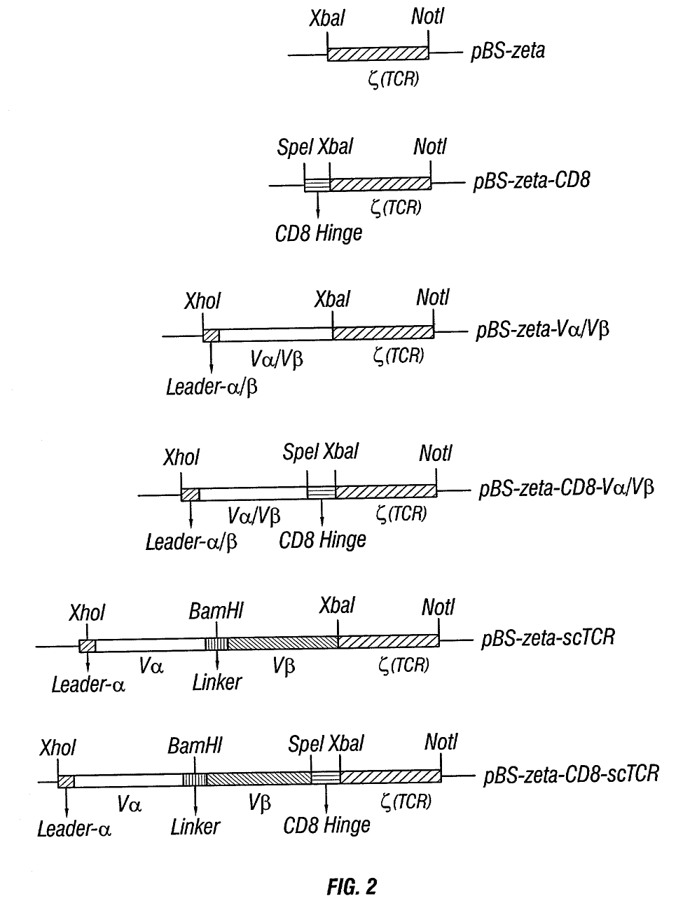

FIGS. 1 and 2 describe some typical derivatives of TCRs useful in the invention. As shown in FIG. 1, a dimeric form may be constructed wherein the variable regions of both α and β chains are directly coupled to the ζ regions of various CD receptors such as CD3, CD8 and CD16. These ζ regions substitute for the transmembrane and cytoplasmic regions normally associated with the TCR. In these examples, the constant region, as it is unnecessary, is eliminated in any case.

Further, in FIG. 1, an alternative construction includes a CD8 hinge region between the variable region and the transmembrane portion of the ζ chain. This spacer may assist in appropriate folding of the receptor. Similarly, in FIG. 1, construction of a single chain TCR wherein the variable regions of the α and β chains are fused through a linker and then fused to the ζ region is shown with and without the CD8 hinge.

FIG. 2 shows a pattern for construction of the relevant plasmids containing the nucleotide sequences encoding the derivatives shown in FIG. 1. As shown hereinbelow, a model system wherein clone 4 TCR directed against hemaglutinin antigen (HA) was used to supply the variable region verified the operability of these approaches.

It is important to recognize that the critical feature of the nucleic acid encoding 2 0 the TCR derivative is the presence of the variable regions from the α and β chains, and that additional sequence, perhaps for added stability, including some or all of the constant region may be present. In addition, alternative transmembrane and signalling regions other than the ζ regions exemplified above may be substituted. Thus, the recombinant materials encoding the TAA-specific, human MHC restricted TCR derivatives of the invention need only include the variable α and β regions of the relevant TCR along with some additional transmembrane and signalling sequence and may further include additional non-interfering amino acid sequence.

The desired CTLs will be specific for TAAs associated with human cancers. Typical among these is Her-2/neu since this proto-oncogene is overexpressed in many human cancers and associated with aggressive disease and malignant transformation (Press, M. S. et al. Cancer Res (1994) 54:5675-5682; Slamon, D. et al. Science (1987) 235:177-182). Other suitable tumor-associated antigens include Ras, p53, tyranase, MART, Gp100, MAGE, BAGE and MUC-1. Any desired antigen which is associated with human tumors can readily be used.

The availability of nucleic acid molecules encoding the desired TCR permits of both diagnostic and therapeutic uses. Cells displaying the TCR at their surfaces can be used as diagnostic for the TAA that is actually expressed by the tumor. In order to conduct such assays, the tumor or a portion thereof or cells derived therefrom are exposed to cells transfected to contain an expression system for the TCR or derivative and the ability of the recombinant CTLs to lyse the tumor cells is assessed. The procedure described in Theobald, M., et al. (1995) supra, may, for example, be used. In addition, an expression for the appropriate TCR may be used therapeutically by transducing such an expression system into the peripheral blood lymphocytes (PBL) CD8⁺ T cells from a tumor-bearing host via, for example, retroviral-mediated gene transfer. Such transfer techniques are known in the art. See, for example, Kasid, A. et al. Proc Natl Acad Sci USA (1990) 87:473, Rosenberg, S. A. et al. New England Journal of Medicine (1990) 323:570. The altered CD8⁺ cells then provide a passive form of immunotherapy. Of course, humanized forms of the TCR as the appropriate derivatives are most helpful in this application.

The following examples are intended to illustrate but not to limit the invention.

### Preparation A

### Model System for TCR Derivatives

Clone 4 TCR (reference) is specific for the hemaglutinin antigen (HA). As the nucleotide sequences encoding the α and β chains of this TCR are available, constructs were made to mimic the intended derivatives of the TAA-specific, HLA-restricted TCR of the invention.

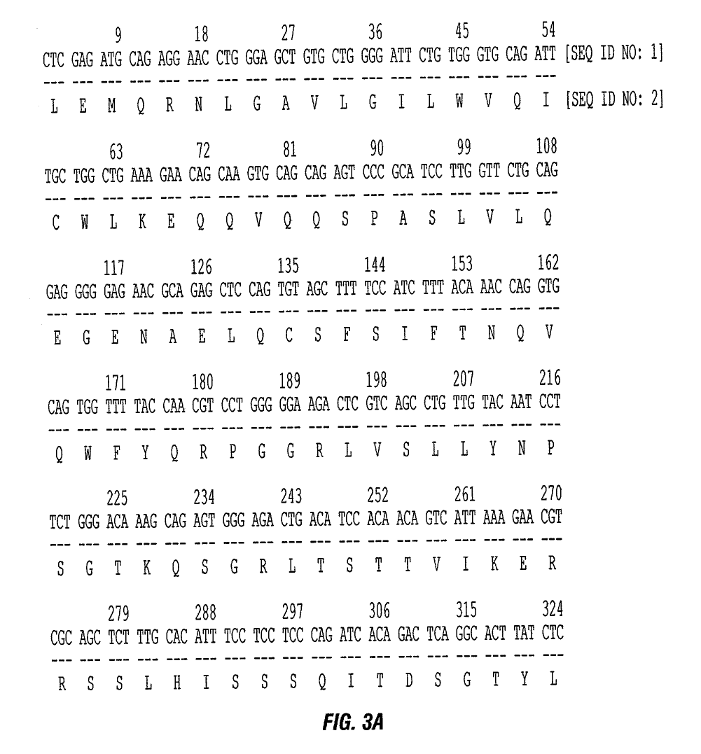

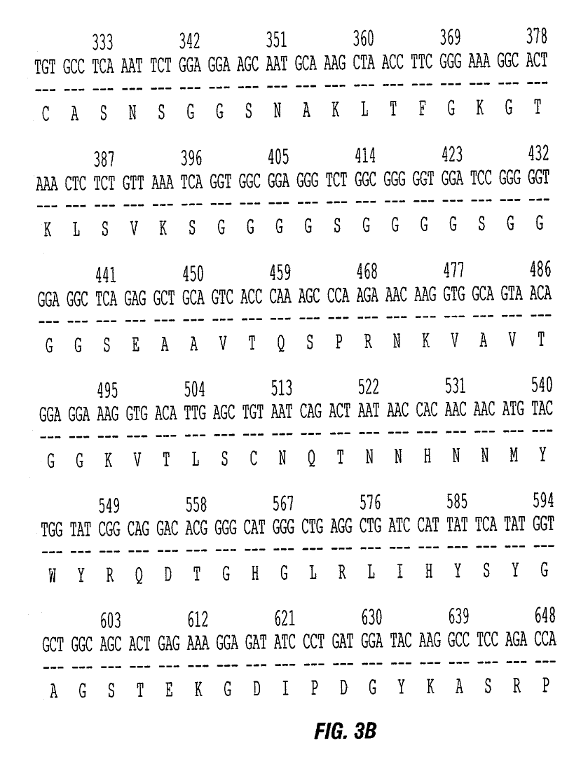

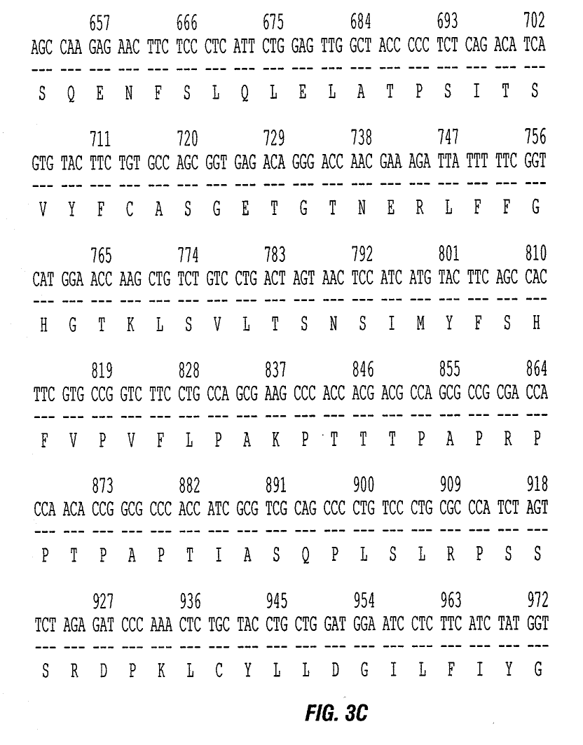

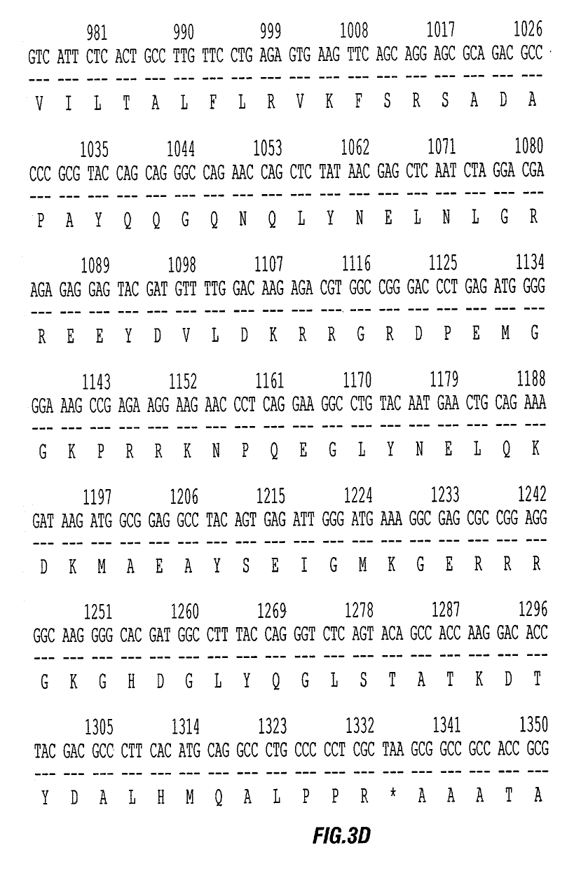

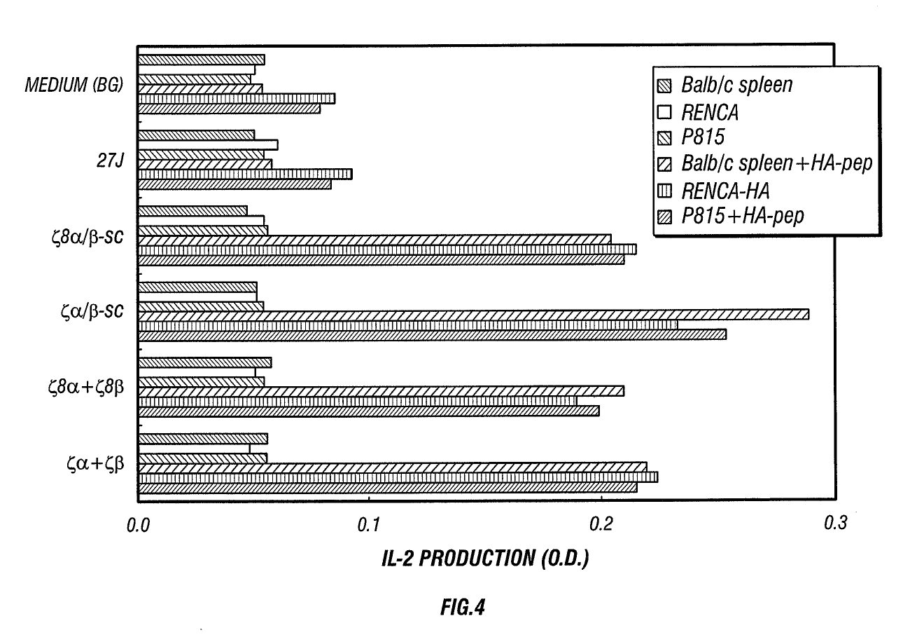

Briefly, four types of chimeric molecules were constructed: two are the dimers obtained as α/ζ+ the β/ζ and two are single-chain TCR/ζ chimeric molecules analogous to those shown in FIG. 1 herein. The complete nucleotide sequence encoding the single chain form with the CD8 hinge is shown in FIGS. 3A-3B. These four constructs were transfected into the T cell hybridoma MD.45-27 and the transformants were grown under neomycin selection and screened for IL-2 secretion upon stimulation with either spleen cells from Balb/c or P815(H-2^{d}) cells pulsed with the HA-specific peptide or RENCA tumor cell line transfected with the HA gene. The results showing the levels of IL-2 produced are shown in FIG. 4. As shown, none of the transfectants showed appreciable production of IL-2 in the absence of HA. Only the transfectants containing the clone 4 derivatives showed stimulation of IL-2 production when HA was present. Both single-chain forms, with and without the CD8 hinge and both dimeric forms, both with and without the CD8 hinge showed appreciable stimulation of IL-2 production when treated either with Balb/c spleen cells plus HA peptide, P815 cells plus HA peptide, or RENCA cells expressing HA at their surfaces.

### EXAMPLE 1

### Selection of Her-2/neu Immunogenic Peptides

Eighteen peptides were synthesized based on the sequence of the human Her-2/neu protein wherein each sequence contained the anchor motif for HLA A2.1, that is, L, I, M, V, A, T at position 2 and position 8/9/10 (Rupert, J. et al. Cell (1993) 74:929-937). The binding efficiency of these peptides to A2 was determined using a competition assay as described by Morrison, J. et al. Eur J Immunol (1992) 22:903-907. Briefly, each test peptide (10 μg) was incubated with radiolabeled target cells (T2-A2.1/K^{b}, 10⁶ target cells labeled with 150 μg ⁵¹Cr at 37° for 1.5 hours) in the presence of an influenza virus matrix protein (0.1 μg). The ability of these peptides to inhibit the binding of the influenza matrix protein peptide M1 (58-66) to A2.1 was measured by inhibition of lysis by an M1 (58-66) specific, A2.1 restricted CTL clone. As shown in Table 1, many of the tested peptides were able to inhibit binding of the M1 peptide.

TABLE 1

Her-2/neu Peptides Used for Immunization

| | | | | |
|---|---|---|---|---|
| | | | IMMUNO- | % |
| PEPTIDE | SEQUENCE # | SEQUENCE | GENICITY | INHIBITION |

| | | | | |
|---|---|---|---|---|
| H3 | 369-377 | KIFGSLAFL | + | 38 |
| H6 | 444-453 | TLQGLGISWL | − | 56 |
| H7 | 773-782 | VMAGVGSPYV | + | 55 |
| H8 | 546-555 | VLQGLPREYV | − | 43 |
| H12 | 48-56 | HLYQGOQW | − | 15 |
| H13 | 689-697 | RLLQETELV | − | 56 |
| H14 | 747-755 | KIPVAIKVL | − | 35 |
| H15 | 789-797 | CLTSTVQLV | − | 33 |
| H16 | 799-807 | QLMPYGCLL | − | 50 |
| H17 | 851-859 | VLVKSPNHV | − | 12 |
| H18 | 871-879 | DIDETEYHA | − | 37 |
| H19 | 933-941 | DLLEKGERL | − | 36 |
| H20 | 971-979 | ELVSEFSRM | − | 5 |
| H21 | 971-980 | ELVSEFSRMA | − | 25 |
| H22 | 972-980 | LVSEFSRMA | − | 14 |
| H23 | 1016-1024 | DLVDAEEYL | − | 35 |
| H24 | 1172-1180 | TLSPGKNGV | − | 57 |
| HIV-9K | POL | KLVGKLNWA | − | 80 |

The peptides were then tested for their ability to elicit an immune response in vivo. The peptides were administered either to A2.1/K^{b}×CD8 or A2.1 transgenic mice and primary cultures of CTLs were generated. Mice were immunized with a mixture of 100 μg of the Her-2/neu peptide with 120 μg ‘helper’ peptide (the helper peptide is a I-A^{b} restricted peptide derived from Hepatitis B virus core protein comprising amino acid residues 128 to 140, that induces a strong CD4 helper response) in 100 μl Incomplete Freuhd's adjuvant. A2.1/K^{b}×CD8 lipopolysacharide (LPS)-blasts were prepared as stimulators for in vitro restimulation of spleen cells from immunized mice. These were prepared by incubating splenocytes in complete RPMI containing 25 μg/ml LPS and 7 μg/ml dextran sulfate at 1.5×10⁶ cells/ml in a total volume of 30 ml for 3 days. Murine spleen cells, collected 10 days after immunization, were restimulated in vitro with the irradiated (3000 rads) blasts which had bound Her-2/neu specific peptides. Six days following in vitro restimulation, the CTL populations were assayed for lytic activity against T2-A2.1/K^{b} target cells preincubated with the peptide used for stimulation (15 μM). The resultant Her-2/neu peptide-specific CTL populations were maintained in vitro by weekly restimulation. CTL populations were restimulated in 2 ml cultures by incubating with 0.1-0.2×10⁶ irradiated Jurkat-A2.1 cells (20,000 rad) preincubated with Her-2/neu peptide (15 μM) and 5×10⁵ irradiated C57BL/6 spleen cells (3000 rad) as fillers in complete RPMI media containing 2% (v/v) supernatent from concanavalin A stimulated rate spleen cells (TCGF).

The cultured cells were assayed for cytotoxicity against T2A2.1/K^{b} target cells pulsed with the priming peptide. In the cytotoxicity assay, 10⁶ target cells were incubated at 37° C. with 150 μCi of sodium ⁵¹Cr chromate for 90 minutes, in the presence or absence of specific peptide. Cells were washed three times and resuspended in 5% RPMI. For the assay, 10^{4 51}Cr-labeled target cells were incubated with different concentrations of effector cells in a final volume of 200 μl in U-bottomed 96 well plates. Supernatants were removed after 4-7 hrs. at 37° C., and the percent specific lysis was determined by the formula: percent specific lysis=100×(experimental release-spontaneous release)/(maximum release-spontaneous release). As shown in Table 1, only the H3 and H7 peptides were able to stimulate a CTL response. (The HIV-9K peptide, known to be immunogenic, was used as a control.)

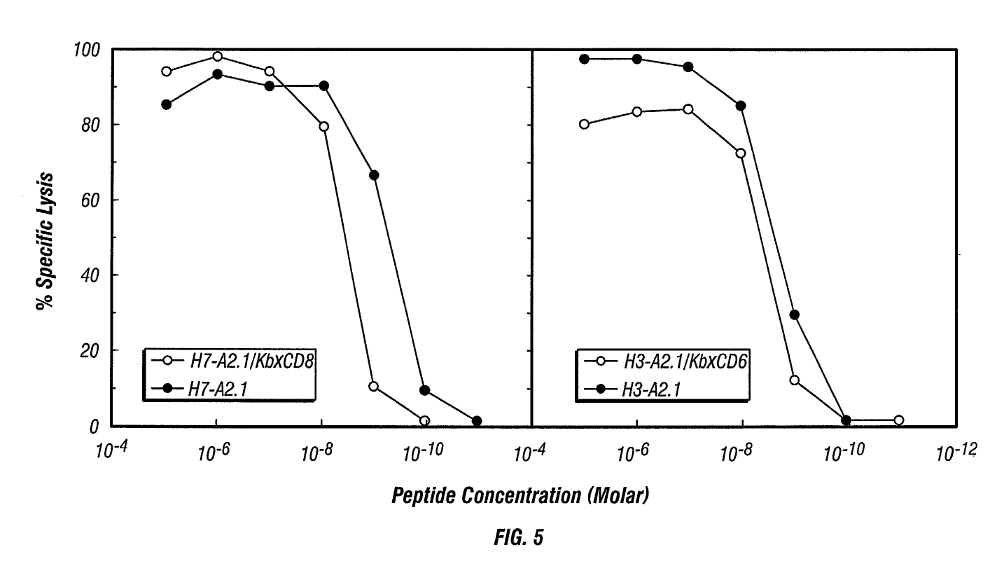

CTL populations that were specific for H3 and H7 were established from either murine strain and maintained in vitro by weekly restimulation. The results of testing these established cell cultures for their ability to lyse T2-labeled targets at a ratio of 1:1 in a four-hour assay in the presence of peptide H3 or H7 are shown in FIG. 5. As shown, the CTLs from either murine subject were comparably effective at comparable peptide concentrations.

### EXAMPLE 2

### Lysis of Human Tumors by H3- and H7-Specific CTL

Various tumor cell lines were characterized by FACS analysis for surface expression of A2 and Her-2/neu peptides. These tumor cells and other control tumors were preincubated or not for 24 hours in media supplemented with 20 ng/ml γ-IFN and 3 ng/ml TNF-α, as such pretreatment increases expression of MHC-1 and adhesion molecules thus enhancing their sensitivity to lysis (Fady, C. et al. Cancer Immuno Immunother (1993) 37:329-336; Fisk, B. et al. Lympho and Cytokine Res (1994) 13:125-131). In the assay, the tumor cells were mixed with the H3- or H7-specific CTL for 6 hours and lysis was measured. HIV-9K-specific CTL were used as a control. The results are shown in Table 2.

TABLE 2
Killing of tumor expressing Her-2/neu

| TUMOR | TYPE | A2 | Her-2 | H7 | H7 + CYT | H3 | H3 + CYT | HIV-9K | HIV-9K + CYT |
|---|---|---|---|---|---|---|---|---|---|
| MDA.MB231 | BREAST | + | + | 26 | 89 | 34 | 85 | 3 | 14 |
| MCF-7 | BREAST | + | + | 7 | 40 | 7 | 54 | 3 | 7 |
| BT549 | BREAST | + | + | 2 | 36 | 2 | 40 | 2 | 15 |
| SAOS.175 | OSTEOSARCOMA | + | + | 27 | 35 | 27 | 33 | 18 | 11 |
| U2-OS | OSTEOSARCOMA | + | + | 30 | 62 | 32 | 91 | 18 | 24 |
| SW480 | COLON | + | + | 2 | 17 | 6 | 50 | 1 | 4 |
| OVCAR-5 | OVARIAN | + | + | 13 | 23 | 25 | 29 | 10 | 12 |
| T98G | GLIOBLASTOMA | + | + | 29 | 93 | 20 | 99 | 9 | 13 |
| MALME-3M | MELANOMA | + | + | 4 | 14 | 28 | 57 | 2 | 1 |
| SKMEL-5 | MELANOMA | + | + | 16 | 40 | 6 | 38 | 5 | 4 |
| NCI.H1355 | LUNG | + | + | 13 | 62 | 11 | 38 | 7 | 25 |
| Hep-G2 | HEPATOMA | + | + | 4 | 29 | 4 | 20 | 1 | 8 |
| CASKI | CERVIX | + | + | 9 | 20 | 13 | 30 | 8 | 11 |
| U87G | GLIOBLASTOMA | + | − | 1 | 1 | 2 | 1 | 5 | 1 |
| ST486 | LYMPHOMA | + | − | 5 | 8 | 1 | 1 | 1 | 1 |
| LG-2 | EBV-TRANS. | + | − | 1 | 3 | 2 | 4 | 1 | 1 |
| SV80 | FIBROBLAST | + | − | 2 | 2 | 4 | 8 | 2 | 2 |
| JY | LYMPHOMA | + | − | 4 | 2 | 2 | 1 | 2 | 1 |
| MDA.MB435 | BREAST | − | + | 1 | 1 | 3 | 2 | 4 | 3 |

As shown, the CTLs were able to lyse effectively only those tumors expressing both A2 and Her-2 peptides. Further, repeating the experiment in the presence of an anti-A2 antibody significantly decreased lysis, and H3 and H7 could be extracted from the tumors using standard techniques.

In a manner similar to that set forth above with respect to H3 and H7, A2-restricted CTLs specific for p53 have been generated. Theobald, M. et al. (1995) (supra).

### EXAMPLE 3

### Recovery of Genes Encoding Her-2/neu and p53 TCRs

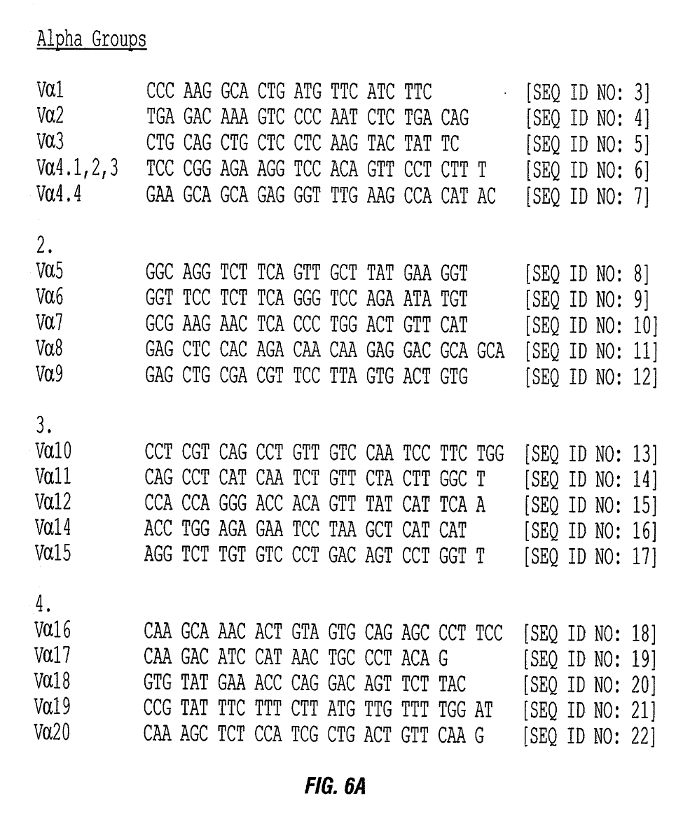

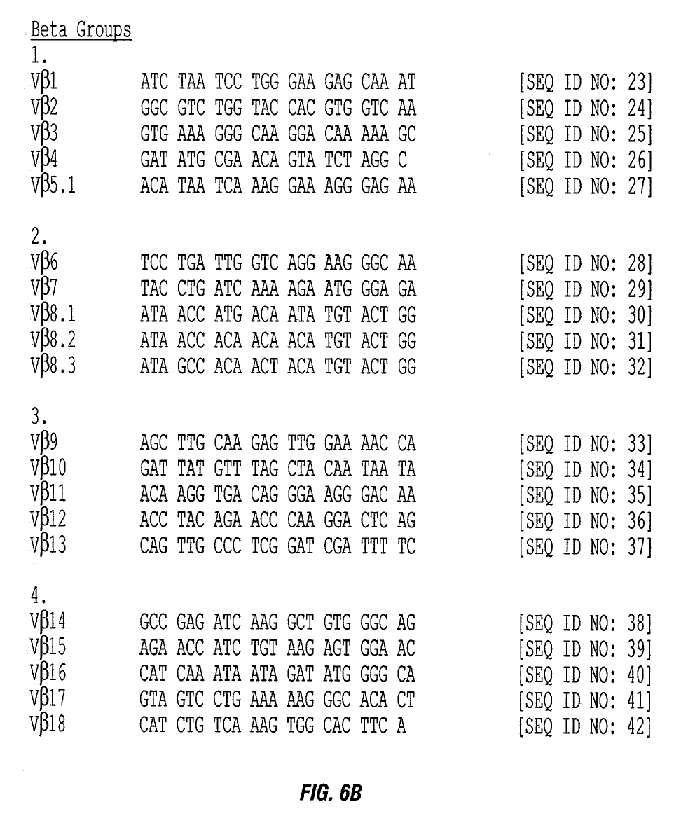

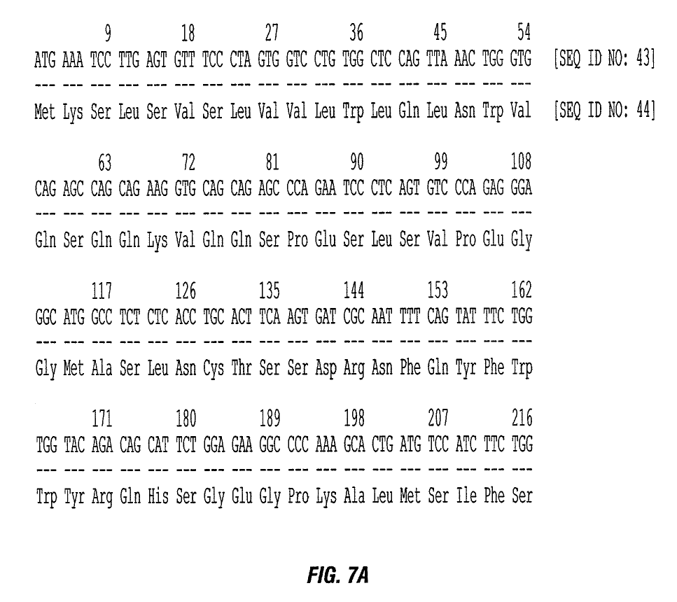

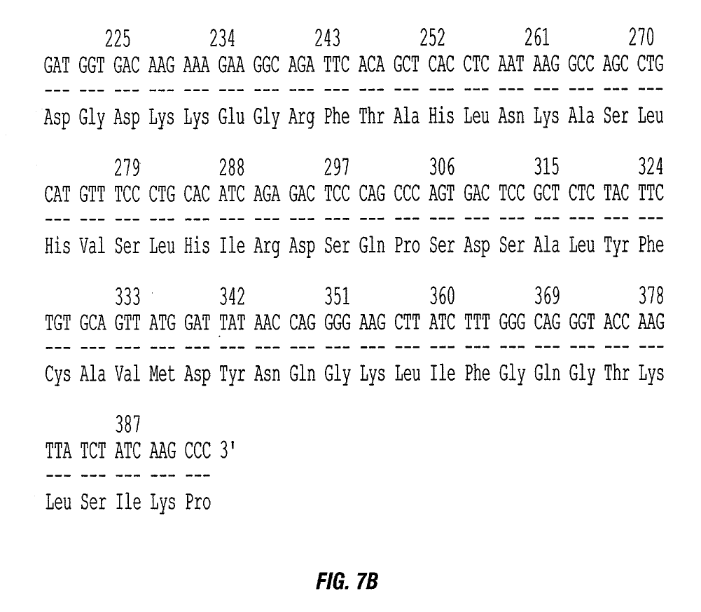

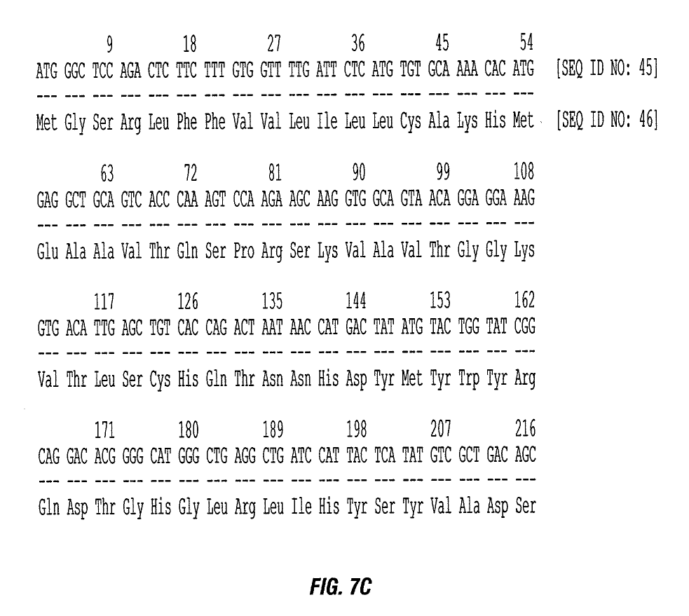

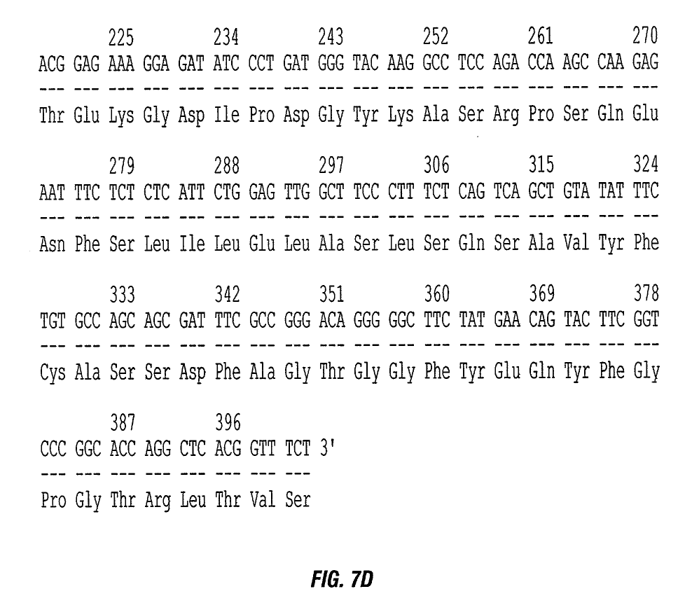

The genes encoding the relevant α and β chains of the TCR specific for H3, H7, and p53 are cloned according to the method of Zisman, B. et al. Eur J Immunol (1994) 24:2497-2505. Primers for the PCR amplification according to these methods are derived from Vα or Vβ families paired with Cα or Cβ primer. Suitable primers for use in this process are shown in FIG. 6. The amplified PCR products are cloned into Bluescript vectors and sequenced. FIG. 7 shows the sequences of the variable regions of the α and β chains of the TCRs recovered from CTLs recovered in mice that had been administered the H7 peptide.

Chimeric molecules similar to those described hereinabove for clone 4 and as set forth in FIGS. 1 and 2 were prepared from the amplified sequences of the H7-specific RR functionality is assayed by transfecting MD45.27 and testing for the production of IL-2 as described hereinabove.

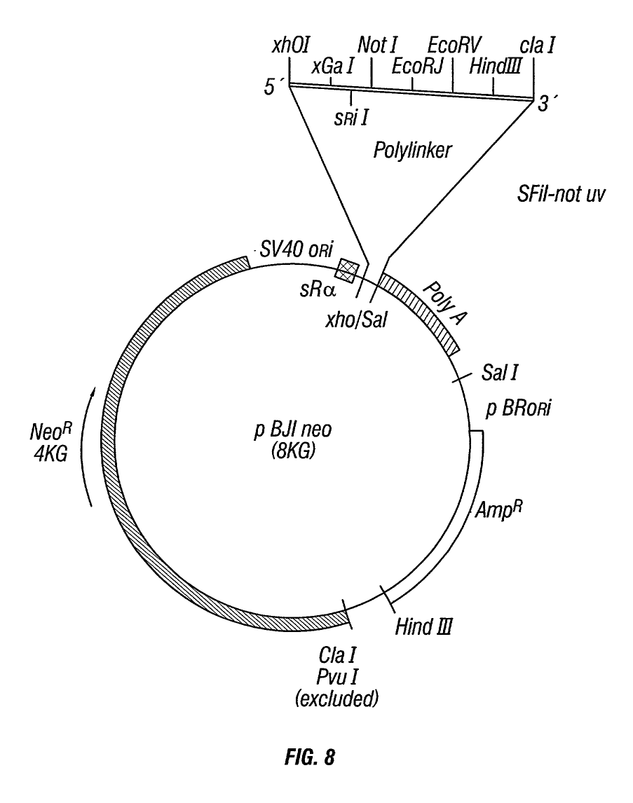

A preferred vector for the insertion of the modified sequences, pBJ1Neo with a polylinker insertion site is shown in FIG. 8. The host vector, pBJINeo is described in ______, Mol Cell Biol (1988) 8:466; the polylinker is described by ______, Science (1990) 249:677.

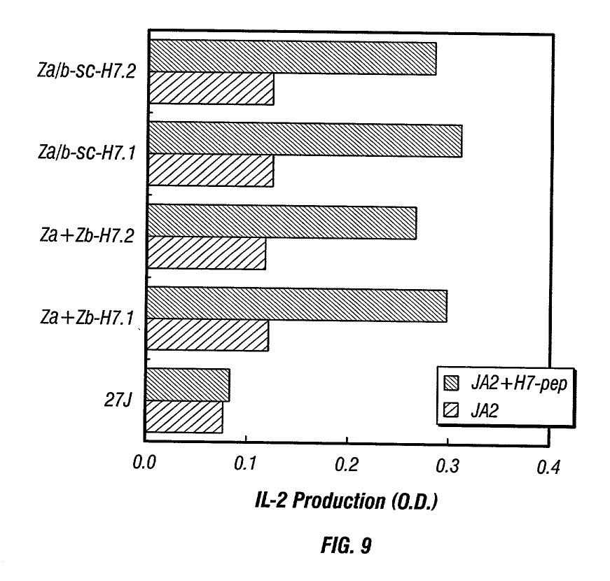

The dimer and single chain constructs were transfected into 27J cells and the cells measured for production of IL-2 in the presence of JA² cells plus H7 peptide. As shown in FIG. 9, all transfectants produced with the H7 specific TCR derivatives produced IL-2. 27J cells without these constructs did not produce IL-2 in response to the JA2 cells and peptide, and none of the cells produced IL-2 in response to JA2 cells alone.

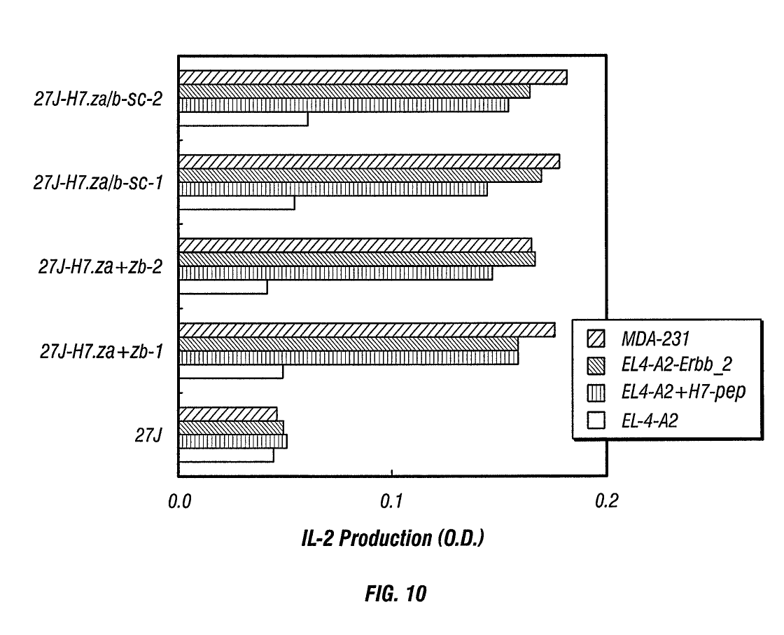

Finally, FIG. 10 shows the production of IL-2 by these four constructs transfected into 27J cells in response to HER 2/neu derived peptides and cells presenting such peptides. Again, all four constructs rendered the transfected cells responsive.

### EXAMPLE 4

### Preparation of T cells Expressing TCR and its Derivatives

Human PBL that are CD8+ are transduced with the chimeric constructs described above using the LXSN and LXSH retroviral vectors (Hock, R. A. et al. Nature (1986) 320:275) and the technique of Anderson, W. F. Science (1992) 256:808. The β chimeric gene is inserted into the LXSH retroviral vector which confers Hygromycin B resistance and α chimeric gene in LXSN retroviral vector which confers neomycin resistance; thus selection of T lymphocytes expressing both the Vα/ζ and Vβ/ζ can be recovered. Recombinant retrovirus-producing cell lines are generated by transfection of the vectors into the Ecotropic packaging cell line GP+E86 and the ecotropic virus produced by these cells is used to infect the amphotropic packaging cell line PA317. PA317 clones that produce helper virus free from amphotropic L(Vα/ζ)SN and L(Vβ/ζ)SH virus are obtained by selection in G418 or Hygromycin B-containing medium. Clones yielding the highest titer of virus are used to transduce T lymphocytes that have been incubated with anti-CD3 and recombinant IL-2. Similarly, the single-chain TCR is inserted into LXSN retroviral vector and introduced similarly.

The resulting transformed human CD8⁺-PBL are tested for cytotoxic activity in vitro against tumor cells and then in vivo in SCID mice that have received tumor cells displaying the relevant TAA.

1. A method to prepare an isolated nucleic acid molecule comprising a nucleotide sequence encoding at least one of the variable regions of the α and β chains of a non-human TCR which TCR is human HLA-restricted and specific for a tumor-associated antigen, which method comprises
cloning or amplifying a nucleic acid molecule containing said encoding nucleotide sequence from cytotoxic T lymphocytes (CTL) prepared by a method which comprises
immunizing a transgenic non-human vertebrate which is modified so as to express at least one human HLA antigen with said tumor-associated antigen (TAA) so as to effect the production in said mouse of cytotoxic T lymphocytes which display human HLA-restricted TCR specific for said TAA and which contain nucleic acid molecules comprising nucleotide sequences encoding said variable regions of the α and β chains of said TCR, and
recovering said CTL.

2. The method of claim 1 wherein said HLA antigen is a A2.

3. The method of claim 1 wherein said non-human vertebrate is a mouse.

4. The method of claim 3 wherein said amplifying is effected by a polymerase chain reaction using primers derived from murine TCR.

5. The method of claim 4 wherein said primers are essentially as set forth in FIG. 6.

6. An isolated nucleic acid molecule which comprises a nucleotide sequence encoding a variable region of a non-human TCR α or β peptide wherein said TCR is human HLA-restricted and specific for a tumor-associated antigen.

7. The nucleic acid molecule of claim 6 which comprises the α or β variable region of the said TCR fused to the ζ region of CD3, CD8 or CD16.

8. The nucleic acid molecule of claim 7 wherein said ζ region is that of human CD3, CD8 or CD16.

9. The nucleic acid molecule wherein said non-human TCR is murine.

10. The nucleic acid molecule of claim 6 wherein said nucleotide sequence encodes a single-chain TCR.

11. The nucleic acid molecule of claim 10 wherein said single-chain TCR consists of the variable α region fused to variable β region by a flexible linker and said β region is fused to a ζ region.

12. The nucleic acid molecule of claim 11 wherein said flexible linker is of the formula (Gly₄Ser₃)₃.

13. The nucleic acid molecule of claim 11 wherein said ζ chain is that of CD3, CD8 or CD16.

14. The nucleic acid molecule of claim 13 wherein the ζ chain is derived from human CD3, CD8 or CD16.

15. A recombinant expression system which expression system comprises the nucleotide sequence of claim 6 operatively linked to control sequences for effecting its expression in a host cell.

16. A recombinant host cell modified to contain the expression system of claim 15.

17. The recombinant cells of claim 16 which are T cells.

18. A method to obtain cells which display TCR or a functional derivative thereof at their surface, said TCR or derivative being human HLA-restricted and specific for a tumor-associated antigen, which method comprises culturing the cells of claim 16 under conditions wherein said nucleotide sequence is expressed and said TCR or derivative is displayed at the surface.

19. Recombinant cells displaying a TCR receptor or derivative thereof at their surface wherein said TCR or derivative is human HLA-restricted and specific for a tumor-associated antigen prepared by the method of claim 18.

20. A method to identify antigens associated with a tumor which method comprises contacting said tumor or a fraction thereof with the cells of claim 19 under conditions wherein said tumor or fraction is lysed only if said tumor displays the TAA for which said TCR or derivative is specific.

21. A method to effect treatment of a tumor in a human, wherein said tumor is characterized by a specific tumor-associated antigen (TAA) which method comprises administering to said human subject peripheral blood cells from said subject which have been modified to contain an expression system for a nucleotide sequence which encodes a TCR or derivative thereof which is human HLA-restricted and specific for said TAA.
